(19) United States
(12) Patent Application Publication
(10) Pub. No.: US 2018/0240554 A1
(43) Pub. Date: Aug. 23, 2018

(54) SYSTEM AND METHOD FOR A BIOMETRIC FEEDBACK CART HANDLE

(71) Applicant: Walmart Apollo, LLC, Bentonville, AR (US)

(72) Inventors: Aaron Vasgaard, Rogers, AR (US); Nicholaus A. Jones, Fayetteville, AR (US); Matthew Jones, Bentonville, AR (US); Kurt William Robert Bessel, Mexico, NY (US)

(73) Assignee: Walmart Apollo, LLC, Bentonville, AR (US)

(21) Appl. No.: 15/902,091

(22) Filed: Feb. 22, 2018

Related U.S. Application Data
(60) Provisional application No. 62/461,933, filed on Feb. 22, 2017.

(51) Int. Cl. G16H 50/30; G06Q 30/00; A61B 5/16

(52) U.S. Cl. CPC G16H 50/30; A61B 5/01; A61B 5/16; G06Q 30/01

(57) ABSTRACT

Systems, methods, and computer-readable storage media for alerting store associates that a customer may need assistance based on biometric data received from the customer via a shopping cart handle. For example, a server may receive, in near-real-time, baseline biometric data generated at a shopping cart handle, the baseline biometric data being associated with a user of the shopping cart. The server may then receive additional biometric data generated at the shopping cart handle and perform an analysis of both the baseline biometric data and the additional biometric data. By this analysis the server can determine that a check on the user should occur, and can transmit a notification to at least one store associate to perform the check on the user.

### BACKGROUND

### 1. Technical Field

The present disclosure relates to identifying customers who may be in need of customer service, and more specifically to identifying those customers based on biometric feedback collected from their shopping cart handle.

### 2. Introduction

As customers shop the aisles of stores, supermarkets, warehouse stores, and other shopping venues, it is common for store associates to ask “Is there anything I can help you with today?” For some customers, this may be a simple but friendly acknowledgement by the store associate of the customer's presence. However, for other customers, such as the sick or elderly, the offer of assistance may be precisely the help they need in navigating the store and collecting their desired items.

### SUMMARY

Disclosed are systems, methods, and non-transitory computer-readable storage media for using biometric data collected from shopping cart handles to identify when customers may need assistance, then sending a notification to a store associate to check on the identified customers.

An exemplary method for performing actions as disclosed herein can include receiving, at a server and in near-real-time, baseline biometric data generated at a shopping cart handle, the baseline biometric data being associated with a user of the shopping cart; receiving additional biometric data generated at the shopping cart handle; analyzing the baseline biometric data and the additional biometric data, to yield a biometric analysis; determining, based on the biometric analysis, that a check on the user should occur; and transmitting a notification to at least one store associate to perform the check on the user.

An exemplary system for performing actions as disclosed herein can include a processor and a computer-readable storage device having instructions stored which, when executed by the processor, cause the processor to perform operations comprising: receiving, in near-real-time, baseline biometric data from sensors at a shopping cart handle, the baseline biometric data being associated with a user of the shopping cart, wherein the baseline biometric data comprises a baseline temperature of the user and a baseline pulse of the user; receiving multiple instances of additional biometric data generated at the shopping cart handle, the additional baseline biometric data comprising an additional temperature of the user and an additional pulse of the user; receiving multiple instances of cart movement data generated at the shopping cart and the shopping cart handle, the cart movement data comprising force applied to the shopping cart handle and shopping cart speed; generating a table of data specific to the shopping cart using the baseline biometric data, the multiple instances of additional biometric data, and the multiple instances of cart movement data; identifying, within the table of data specific to the shopping cart, an indicator that the user may need assistance, to yield an identification; and transmitting, based on the identification, a notification to at least one store associate to perform a check on the user.

An exemplary non-transitory computer-readable storage medium for performing actions as disclosed herein can have instructions stored which, when executed by a computing device, cause the computing device to perform operations comprising: receiving, at a server and in near-real-time, baseline biometric data generated at a shopping cart handle, the baseline biometric data being associated with a user of the shopping cart; receiving additional biometric data generated at the shopping cart handle; analyzing the baseline biometric data and the additional biometric data, to yield a biometric analysis; determining, based on the biometric analysis, that a check on the user should occur; and transmitting a notification to at least one store associate to perform the check on the user.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

### BRIEF DESCRIPTION OF THE DRAWINGS

FIG. 1 illustrates the interactions between a customer, a shopping cart, a server, and a store associate;

FIG. 2 illustrates a first example method;

FIG. 3 illustrates interconnectivity between a server, an antenna, a database and a call system;

FIG. 4A illustrates an example of baseline data;

FIG. 4B illustrates an example of post-baseline data;

FIG. 5 illustrates collected data over time;

FIG. 6 illustrates a second example method; and

FIG. 7 illustrates an exemplary system embodiment.

### DETAILED DESCRIPTION

A system, method and computer-readable media are disclosed which allow for store associates to be notified that a customer may need assistance based on biometric data collected from shopping cart handles as the customer is shopping. Consider the following example. A customer walks into a store and selects a shopping cart. The shopping cart, upon being moved, “wakes up” from being in a low-power, or “sleep” state, and begins procedures to collect baseline biometric data from the customer using sensors embedded in the shopping cart handle.

Non-limiting examples of the baseline biometric data collected by the shopping cart can include the customer's heart rate, the customer's temperature, the force of the grip of the customer, and how much force the customer is applying to the cart (i.e., how hard the customer is leaning into the cart, or how hard the customer is pushing the cart). The shopping cart transmits this data to a server, which in turn stores and analyzes the biometric data received. In some configurations, the server is located in the store and is uniquely configured for that store, whereas in other configurations the server can be remotely located. In yet other configurations, the server can be a cloud-based server or cloud-based computing system.

The server receives the baseline biometric data and records the baseline biometric data as a new customer. As the shopping cart is pushed around the store by the customer, the shopping cart collects additional biometric data and transmits that data to the server. For example, the shopping cart sensors can update the metrics recorded during the baseline reading, then transmit the updated biometric values to the server as additional biometric data. In addition, as the customer moves around the store, the shopping cart can record cart movement data, such as the cart speed, store temperature, force being applied to the shopping cart handle, weight of products in the cart, noise level in the store, cart location, and the like. The additional biometric data and the cart data can be sent to the server at regular (periodic) intervals, or can be sent to the server upon trigger conditions being met.

The server can, over time, build a table of the data associated with a customer's visit to the store, the table being made of the biometric data and/or the non-biometric cart data. Specifically, as the data is received the data can be recorded and added to the table. In addition, within the table can be values and data extrapolations based on the other data. For example, the server can create, within the table, a metric for the customer's stress. This stress estimate can, for example, be calculated by weighting the biometric and non-biometric factors. As one example, if a customer's temperature is increasing while the customer's grip on the shopping cart handle simultaneously increases in force, the stress estimate may increase. In another example, the stress estimate may increase based on slow or limited movement of the shopping cart, such as when the customer is waiting in line to checkout or in a busy portion of the store.

As the data is being received (i.e., in real-time, or near real-time), the server can perform analyses on the table of data being compiled. This real-time analysis of the table identifies trends in the data indicating the user may need assistance by determining that the biometric data received exceeds a range (an upper and lower threshold) of acceptable data. In some cases, this range can be predefined. For example, the range of acceptable body temperatures of the customers may be 97.5° F.-99.5° F. When the customer enters the store, the baseline temperature measurement may measure the customer's temperature at 99° F., but after a few minutes of shopping the biometric sensors in the shopping cart handle measure the customer's temperature at 101° F. Because the 101° F. exceeds the predefined range, the server can flag the customer's temperature and dispatch a store associate to check on the customer.

In other configurations, the acceptable ranges can be defined based on the baseline data received when the customer first begins using the cart. For example, if (as in the previous example) the customer had a baseline temperature of 99.3° F., the server can set the range at 98.3° F.-100.3° F., thereby adjusting the acceptable range to plus or minus 1 on the measured temperature. In yet other configurations, the ranges can be a combination of predefined and customer-specific elements. As an example, the system can have predefined ranges of acceptable heart rates as 60-120 beats per minute (bpm), where any measurement outside of that predefined range automatically triggers a store associate notification. However, the system can also have a customer-specific range based on the heart rate recorded in the baseline measurement, where measurements outside the customer-specific range likewise trigger store associate notification.

In addition to identifying the need for customer assistance based on use of ranges, the server can identify patterns within the collected data which indicate the customer may need help. For example, in normal use, force initially applied to move the cart increases as the inertia of the cart is overcome, then levels off as the cart is pushed through the store. Deviating from this pattern may indicate the customer needs assistance. An exemplary pattern which may appear if the customer is struggling to push the cart is that the force applied to the shopping cart handle may have a rapid, periodic pattern of the user pushing, but without any resulting cart speed. Another exemplary pattern the customer may need assistance can be abrupt changes in the cart speed (possibly indicating that the customer is crashing the cart to stop it).

When the server has identified conditions indicating the customer may need assistance, the server can send a signal to a store associate (such as a store worker, a store manager, etc.) to check on the customer. Often this check can be a simple “Can I help you find anything?” or “Do you need any help?” To transmit the notification to the store associate, the server can operate with an Associate Call System. Such a system can, for example, contain information for contacting the individual store associates using wireless or radio signals. For example, the server can (having identified that an associate should check on a customer) identify, from the Associate Call System, the necessary information to contact a specific employee, prepare the message, and send the message with the contact information to an antenna, a wireless router, or other transmission system. The notification sent from the server via the antenna or wireless router is then received by device associated with the store associate. Examples of such devices can include a telephone, a beeper, a mobile phone, a smartphone, a tablet, a handheld device (such as a Telxon, MC 40, MC 55, etc.), and/or a smart wearable (such as a smart watch, smart glasses, etc.).

If certain conditions are met, a notification may be sent over the store intercom. For example, if the customer's biometric data indicates that the customer is possibly in need of immediate attention, a notification can be broadcast over the store intercom. In such circumstances, certain configurations can also allow for notifying of medical personnel. To determine if the conditions for such a notification have been met, the system uses the same analysis processes described above.

In some configurations, the server can identify real-time trends in the analyses across multiple customers. For example, the server may identify that the heart rate of most customers increases when they enter certain aisles or portions of the store, when certain music is playing across the intercom, when too many customers are in a single portion of the store, or when specific store associates are nearby. This data can then be communicated to store management, who can take action to improve the shopping experience. In addition, if the analyses indicate that multiple customers in a single portion of the store are all, within a narrow timeframe, needing to be checked on, this may indicate that there is a larger problem (such as a spill, a smell, a customer dispute, or other issue), and can initiate a measured response. For example, the server could dispatch multiple store associates or the store manager to that portion of the store to determine what is causing the multiple customers to (at least from the perspective of the server) simultaneously require additional assistance.

It is noted that the biometric data and the cart movement data collected during the use of the shopping cart is not tied or otherwise linked to the identity of the individual customer. When carts are idle, before being selected by a customer for the shopping experience, the cart contains no information about actual or potential customers. Once selected, the biometric sensors within the shopping cart handle make the appropriate measurements and transmit that information to the server, where a new table of data associated with the selected shopping cart is generated, with new data being added to the table throughout the shopping experience. When the customer is finished shopping, the cart can determine that the shopping experience has ended based on factors such as the cart no longer moving, the lack of force being applied to the shopping cart handle, the weight of the cart, the cart location, etc. Upon making that determination, the shopping cart can enter a sleep mode to await the next customer. At the same time, the server can identify that the cart has entered a sleep mode and delete the any correlation between the table of the customer's biometric data and the specific cart which was used. The table can then be used in conjunction with data received from other carts to better predict behavior and needs of customers.

These concepts and features of the disclosure are further described below in conjunction with the illustrations. While specific implementations are described, it should be understood that this is done for illustration purposes only. Other components and configurations may be used without parting from the spirit and scope of the disclosure.

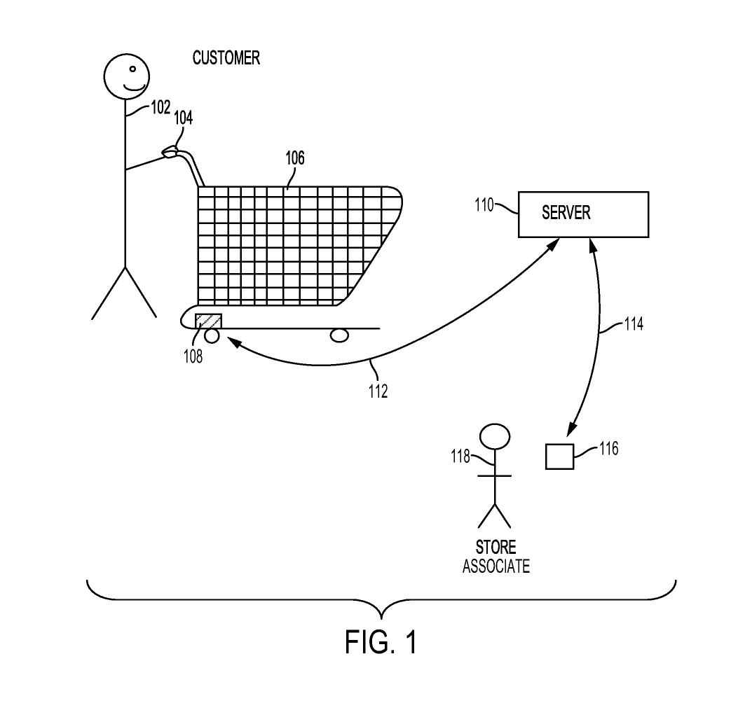

FIG. 1 illustrates the interactions between a customer 102, a shopping cart 106, a server 110, and a store associate 118. As the customer 102 holds onto the shopping cart handle 104, biometric data of the customer 102 is detected by biometric sensors within the shopping cart handle 104. This biometric data is transmitted 112 from the shopping cart 106, via a communications module 108, to a server 110. Such transmission 112 of data is performed wirelessly using, for example, Wi-Fi, Radio, or Bluetooth signals. The server 110 receives the data, stores the data, processes the data, and performs analyses on the data as described above. Upon making the determination that a store associate 118 should be notified to check on the customer 102, the server transmits a signal 114 to a mobile device 116 of the store associate 118.

FIG. 1 shows only a single customer 102 utilizing the shopping cart 106. However, often multiple individuals are shopping together using a single cart. Detection of multiple, distinct users using a single cart can occur by recording the biometric data at distinct times and noting threshold distinctions. For example, a first user may have a heart rate of 65 bpm, and the second user a heart rate of 90 bpm. As the users swap holding the handle of the cart, the distinctions between the users can be identified. In such instances, the server 110 can maintain multiple tables tied to the respective individuals, such that when User A is moving the cart data associated with User A is recorded into a first table, and when User B is moving the cart data associated with User B is recorded into a second table. Analyses can then be run by the server 110 on both tables as they are respectively refreshed with updated biometric and cart data.

Also in FIG. 1, the communications module 108 is illustrated as being distinct from the shopping cart handle 104. However, in some configurations the shopping cart handle 104 can have capabilities to wirelessly transmit data 112 to the server 110 from within the shopping cart handle 104. In other configurations, the shopping cart 106 can have communication capabilities built into the cart itself. In addition, FIG. 1 illustrates the processing and analyses of the biometric data being done by a server 110. However, in some configurations the software required for such analyses can be present in the shopping cart handle 104, with notifications to store associates 118 being generated at the shopping cart 106 and transmitted directly therefrom.

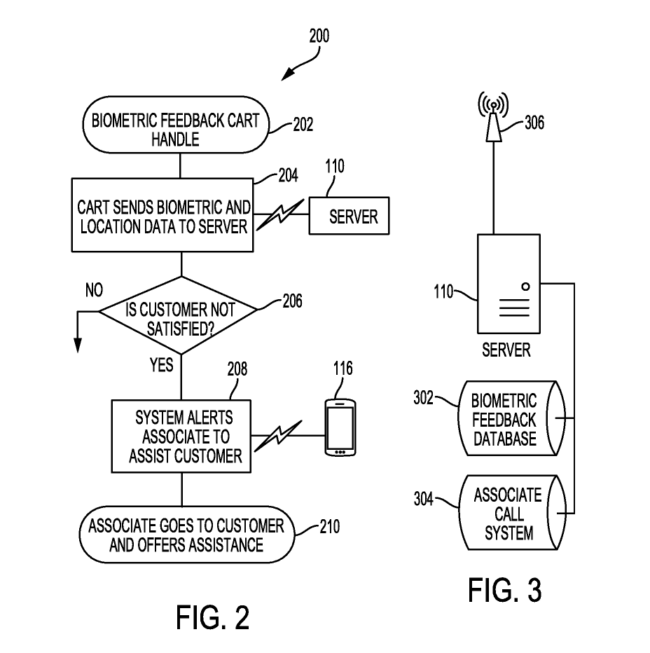

FIG. 2 illustrates a first example method 200, outlining the processes being discussed herein. In this example 200, a biometric feedback cart handle (202) is used by a customer 102, at which point the cart 106 sends biometric and location data (204) to a server 110. With each communication, the server 110 determines if the customer 102 is dissatisfied (206). If the customer 102 is not dissatisfied (that is, if the customer 102 is content), this process of collecting the biometric and location data (204) continues. If, however, the customer 102 is dissatisfied, the system alerts an associate 118 to assist the customer (208). This alert transmits a signal to, for example, a mobile device 116 of the associate 118. The associate 118 then goes to the customer 102 and offers assistance (210).

FIG. 3 illustrates interconnectivity between a server 110, an antenna 306, a database 302, and a call system 304. The server 110 discussed herein receives and transmits data using an antenna 306. As data is received from the shopping carts 106 in a store, the server 110 records such data in a biometric feedback database 302. For example, the biometric feedback data can be recorded on tables associated with each shopping cart 106 currently in use in the store. When the server 110 determines that an associate 118 should be notified that a customer needs assistance, the server 110 can communicate with an associate call system 304. The associate call system 304 can contain contact information for the associates 118 in the store, such as the identification information associated with the mobile devices 116 of the associates 118.

As illustrated, the antenna 306 is distinct from the server 110. Likewise, the biometric feedback database 302 and the associate call system 304 are illustrated as distinct components. However, in other configurations the antenna 306, the biometric feedback database 302, and/or the associate call system 304 may be incorporated into the server 110.

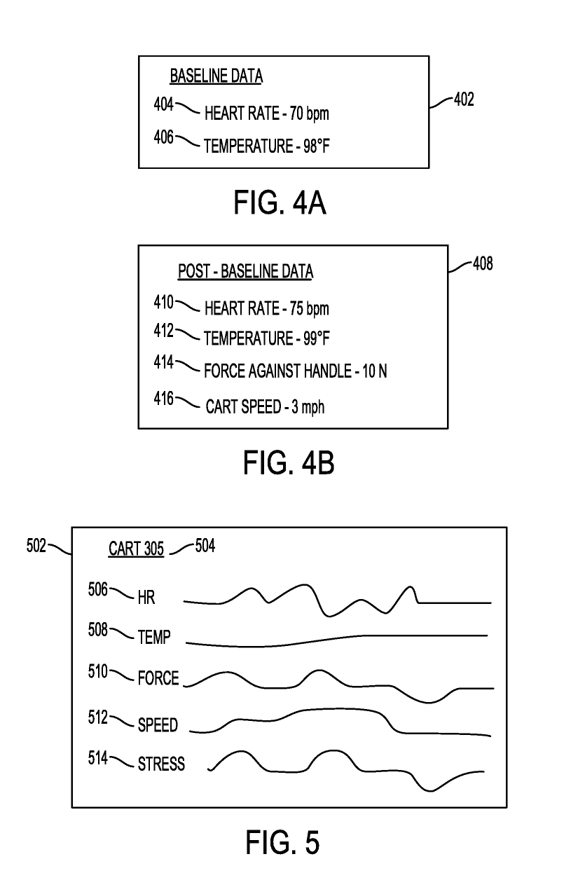

FIG. 4A illustrates an example of baseline biometric data 402 which can be detected by the cart handle 104 sensors. In this example, the baseline biometric data 402 includes heart rate 404 and temperature 406 of the customer 102. In other configurations, the baseline data can contain fewer or more elements. Additional, or alternative, elements which can be collected as baseline biometric data 402 include palm humidity (sweatiness of the hands), oxygen absorption, softness of the hands, and/or size of the hands.

FIG. 4B illustrates an example of post-baseline data 408. This data 408 includes both additional biometric data 410, 412 as well as cart movement data 414, 416. The additional biometric data 410, 412 represents updated values to the baseline data 402 which, as illustrated, is updated heart rate 410 and updated temperature 412 information. The exemplary cart movement data illustrated in FIG. 4B is the force being applied 414 against the shop cart handle and the cart speed 416. Additional cart movement data which can be collected includes the cart location, the weight of the items in the cart, the noise level at the cart, and wheel status reports (i.e., if any of the wheels are not turning properly).

FIG. 5 illustrates collected data over time 502. In this illustration 502, the data which has been collected into a table for an individual cart 504 is presented in graphical form for easier interpretation by human beings. Under normal operations, the server 110 will not need to generate such a graphical form unless requested to by a supervising user. However, whereas the server 110 can easily interpret trends within a table of information (and draw conclusions from that table), for most individuals the collected data is more easily understood in graphical form. In this illustration 502, heart rate 506, temperature 508, force on the cart handle 510, cart speed 512, and estimated stress of the user 514 are all presented in graphical form. From the data used to generate such a graphical interpretation, the server 110 can identify if the customer 102 has crossed any defined thresholds or otherwise produced patterns indicating the customer should be offered assistance.

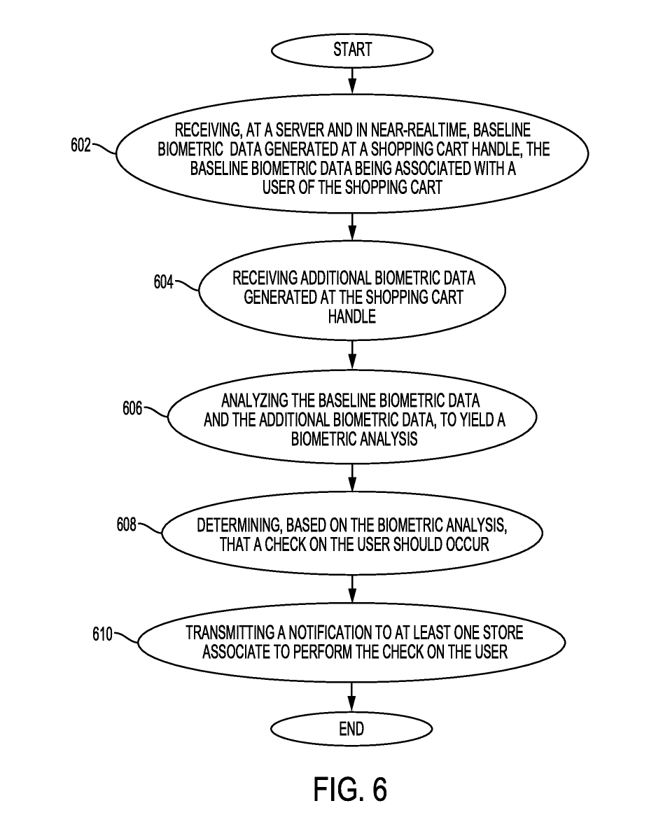

FIG. 6 illustrates a second example method, performed by the server 110 of FIG. 1. The method includes receiving, at the server 110 and in near-real-time, baseline biometric data generated at a shopping cart handle 104, the baseline biometric data being associated with a user 102 of the shopping cart 106 (602). The server 110 receives additional biometric data generated at the shopping cart handle 104 (604) and analyzes the baseline biometric data and the additional biometric data, to yield a biometric analysis (606). The server 110 then determines, based on the biometric analysis, that a check on the user 102 should occur (608). The server 110 then transmits a notification to at least one store associate 118 to perform the check on the user 102.

Examples of the biometric data which can be collected can include the temperature and the heart rate of the user 102. The biometric analysis can include determining, based on the baseline biometric data, a baseline range; recording multiple instances of the additional biometric data over time, to yield a table of biometric data; identifying, within the table of biometric data, values which fall outside the baseline range; and based on the values being outside the baseline range, determining that the check on the user should occur.

Receiving of the additional biometric data and/or the cart data can occur at regular, periodic intervals. Alternatively, the additional biometric data and/or the cart data can occur based upon trigger conditions being met at the shopping cart. For example, every time the shopping cart detects a new item has been added to the cart, a measurement can be taken immediately afterwards. As another example, the shopping cart may take biometric measurements each time the cart moves after being stopped for more than 3 seconds (or another predetermined period of time). As yet another example, measurements can be taken each time the shopping cart enters a new aisle, or when the shopping cart enters into a new section/portion of the store.

The method of FIG. 6 can also include receiving cart movement data, the cart movement data including the force applied to the shopping cart handle and the shopping cart speed. In addition, the server 110 can use the received data to generate additional fields, such as a stress estimation of the user. Such additional fields can be created by weighting the known data. For example, a simple estimation of the user's stress may be Stress=heart rate×force of the grip on the handle bar. More complex estimation of the user's satisfaction may include the user's temperature, the user's speed in the store, the store noise level, etc., all weighted as required by specific configurations. One example would be that the location of the cart continues to be at a certain location when the humidity sensor senses extra moisture and the force on the cart handle increases. This could lead to a determination to send help to that location repeatedly, which can be used to determine that store customers do not like that area of the store. Likewise, analysis of the increase in speed through force on the handle and/or an elevated heart rate, can be used to determine an area of the store customers do not like.

In some configurations, the server 110 can also do group analysis on carts being used in the store. For example, using the data collected from multiple users, the server 110 can identify, identifying, within a portion of the store in which the shopping cart is located, additional shoppers who, based on biometric data received from shopping cart handles of shopping carts used by the additional shoppers, should also be checked on, to yield an identified pattern; and based on the identified pattern, sending an additional notification to the at least one store associate, the additional notification identifying the portion of the store in which the identified pattern is occurring. This type of group analysis can reveal trends among multiple customers that something in that portion of the store needs to be addressed.

In other embodiments, biometric feedback can be provided to the customer via, for example, a smartphone application. The items placed into the cart may be tracked, for example, by scanning a code such as the bar code or UPC on an item. The system can determine the items placed in the cart, the weight of the items and the force applied to the cart, speed and distance traveled. Based on the collected data, the system can determine the work being done by pushing the cart. The speed, mileage, calories burned, etc. may be provided to the customer via the smartphone application.

The customer's heart rate may also be tracked and provide to the customer. Based on the customer's age, weight of the cart and items, feedback may be provided to advise the customer to slow down. Data regarding the heart rate, age may be stored in various database tables. The tables are consulted to provide the feedback to the customer.

In another embodiment, the cart may be provided with a power assist mode. When the cart is over a certain weight, the power assist may be activated. The heart rate and other variable may be monitored to activate the power assist to keep the parameters in the desired range. The system may determine the proper ranges based on the data previously collected, or for the data for the particular customer.

In another embodiment, a timer may be provided. The timer measure an amount of time the customer has let go of the cart handle. When the time exceeds a predetermined time, an alert may be issued to check on the customer.

In another embodiment, a pulse oximeter may be provided. The pulse oximeter may be used to measure the customer's oxygen saturation. Acceptable ranges for the customer may be stored by the system. The ranges may be based on general characteristic or for the particular characteristics for the customer. The oxygen saturation may be monitored to determine if the customer is at risk for passing out or other incident. Based on a comparison of the measure oxygen saturation and the stored ranges, assistance may be dispatched to the customer.

Any combination or sub combination of the above discussed parameters may be measured, tracked, and provided to the customer.

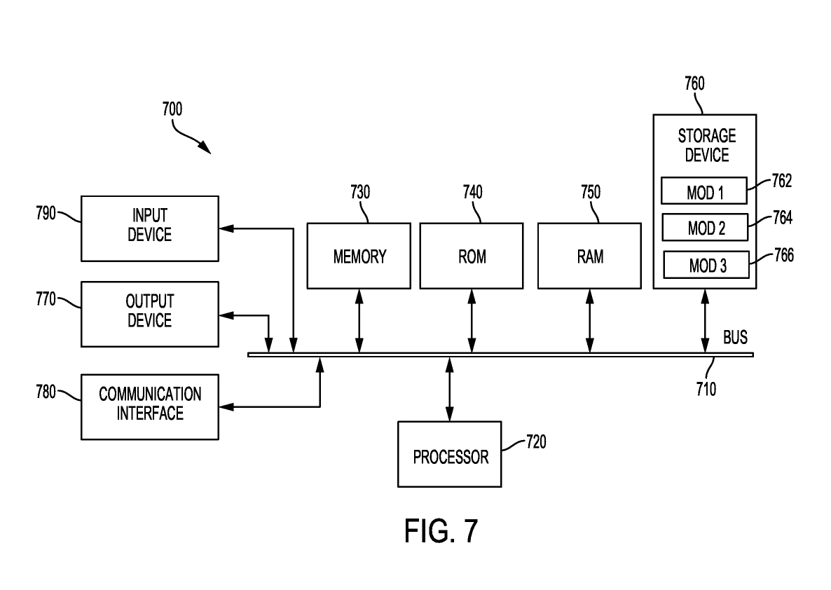

With reference to FIG. 7, an exemplary system 700 includes a general-purpose computing device 700 which can be employed to practice the concepts disclosed herein, including a processing unit (CPU or processor) 720 and a system bus 710 that couples various system components including the system memory 730 such as read only memory (ROM) 740 and random access memory (RAM) 750 to the processor 720. The system 700 can include a cache of high speed memory connected directly with, in close proximity to, or integrated as part of the processor 720. The system 700 copies data from the memory 730 and/or the storage device 760 to the cache for quick access by the processor 720. In this way, the cache provides a performance boost that avoids processor 720 delays while waiting for data. These and other modules can control or be configured to control the processor 720 to perform various actions. Other system memory 730 may be available for use as well. The memory 730 can include multiple different types of memory with different performance characteristics. It can be appreciated that the disclosure may operate on a computing device 700 with more than one processor 720 or on a group or cluster of computing devices networked together to provide greater processing capability. The processor 720 can include any general purpose processor and a hardware module or software module, such as module 1 762, module 2 764, and module 3 766 stored in storage device 760, configured to control the processor 720 as well as a special-purpose processor where software instructions are incorporated into the actual processor design. The processor 720 may essentially be a completely self-contained computing system, containing multiple cores or processors, a bus, memory controller, cache, etc. A multi-core processor may be symmetric or asymmetric.

The system bus 710 may be any of several types of bus structures including a memory bus or memory controller, a peripheral bus, and a local bus using any of a variety of bus architectures. A basic input/output (BIOS) stored in ROM 740 or the like, may provide the basic routine that helps to transfer information between elements within the computing device 700, such as during start-up. The computing device 700 further includes storage devices 760 such as a hard disk drive, a magnetic disk drive, an optical disk drive, tape drive or the like. The storage device 760 can include software modules 762, 764, 766 for controlling the processor 720. Other hardware or software modules are contemplated. The storage device 760 is connected to the system bus 710 by a drive interface. The drives and the associated computer-readable storage media provide nonvolatile storage of computer-readable instructions, data structures, program modules and other data for the computing device 700. In one aspect, a hardware module that performs a particular function includes the software component stored in a tangible computer-readable storage medium in connection with the necessary hardware components, such as the processor 720, bus 710, display 770, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 700 is a small, handheld computing device, a desktop computer, or a computer server.

Although the exemplary embodiment described herein employs the hard disk 760, other types of computer-readable media which can store data that are accessible by a computer, such as magnetic cassettes, flash memory cards, digital versatile disks, cartridges, random access memories (RAMs) 750, and read only memory (ROM) 740, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 700, an input device 790 represents any number of input mechanisms, such as a microphone for speech, a touch-sensitive screen for gesture or graphical input, keyboard, mouse, motion input, speech and so forth. An output device 770 can also be one or more of a number of output mechanisms known to those of skill in the art. In some instances, multimodal systems enable a user to provide multiple types of input to communicate with the computing device 700. The communications interface 780 generally governs and manages the user input and system output. There is no restriction on operating on any particular hardware arrangement and therefore the basic features here may easily be substituted for improved hardware or firmware arrangements as they are developed.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure.

We claim:

1. A method comprising:
receiving, at a server and in near-real-time, baseline biometric data generated at a shopping cart handle, the baseline biometric data being associated with a user of the shopping cart;
receiving additional biometric data generated at the shopping cart handle;
analyzing the baseline biometric data and the additional biometric data, to yield a biometric analysis;
determining, based on the biometric analysis, that a check on the user should occur; and
transmitting a notification to at least one store associate to perform the check on the user.

2. The method of claim 1, further comprising:
receiving cart movement data, the cart movement data comprising force applied to the shopping cart handle and shopping cart speed.

3. The method of claim 1, wherein in the baseline biometric data and the additional biometric data each respectively comprise a pulse of the user and a temperature of the user.

4. The method of claim 1, wherein the biometric analysis comprises:
determining, based on the baseline biometric data, a baseline range;
recording multiple instances of the additional biometric data over time, to yield a table of biometric data;
identifying, within the table of biometric data, values which fall outside the baseline range; and
based on the values being outside the baseline range, determining that the check on the user should occur.

5. The method of claim 4, wherein the multiple instances of the additional biometric data are received at regular intervals.

6. The method of claim 4, wherein the multiple instances of the additional biometric data are received based upon a trigger condition being met at the shopping cart.

7. The method of claim 4, wherein the biometric analysis further comprises:
generating, within the table of biometric data, a stress estimation, wherein the stress estimation is generated by weighting the baseline biometric data and the additional biometric data.

8. The method of claim 1, further comprising:
identifying, within a portion of the store in which the shopping cart is located, additional shoppers who, based on biometric data received from shopping cart handles of shopping carts used by the additional shoppers, should also be checked on, to yield an identified pattern; and
based on the identified pattern, sending an additional notification to the at least one store associate, the additional notification identifying the portion of the store in which the identified pattern is occurring.

9. A system, comprising:
a processor; and
a computer-readable storage device having instructions stored which, when executed by the processor, cause the processor to perform operations comprising:
receiving, in near-real-time, baseline biometric data from sensors at a shopping cart handle, the baseline biometric data being associated with a user of the shopping cart, wherein the baseline biometric data comprises a baseline temperature of the user and a baseline pulse of the user;
receiving multiple instances of additional biometric data generated at the shopping cart handle, the additional baseline biometric data comprising an additional temperature of the user and an additional pulse of the user;
receiving multiple instances of cart movement data generated at the shopping cart and the shopping cart handle, the cart movement data comprising force applied to the shopping cart handle and shopping cart speed;
generating a table of data specific to the shopping cart using the baseline biometric data, the multiple instances of additional biometric data, and the multiple instances of cart movement data;
identifying, within the table of data specific to the shopping cart, an indicator that the user may need assistance, to yield an identification; and
transmitting, based on the identification, a notification to at least one store associate to perform a check on the user.

10. The system of claim 9, wherein the computer-readable storage device contains additional instructions which, when executed by the processor, cause the processor to perform additional operations comprising:
generating, within the table of data specific to the shopping cart, a stress estimation of the user, the stress estimation based on the baseline biometric data, the multiple instances of additional biometric data, and the multiple instances of cart movement data.

11. The system of claim 9, wherein the indicator is based on a metric within the table of data specific to the shopping cart exceeding a range.

12. The system of claim 11, wherein the range is customized to the user based on the baseline biometric data, the multiple instances of additional biometric data, and the multiple instances of cart movement data.

13. The system of claim 9, wherein the multiple instances of additional biometric data are received at regular intervals.

14. The system of claim 9, wherein the multiple instances of additional biometric data are received based upon a trigger condition being met at the shopping cart.

15. A non-transitory computer-readable storage medium having instructions stored which, when executed by a computing device, cause the computing device to perform operations comprising:
receiving, at a server and in near-real-time, baseline biometric data generated at a shopping cart handle, the baseline biometric data being associated with a user of the shopping cart;
receiving additional biometric data generated at the shopping cart handle;
analyzing the baseline biometric data and the additional biometric data, to yield a biometric analysis;
determining, based on the biometric analysis, that a check on the user should occur; and
transmitting a notification to at least one store associate to perform the check on the user.

16. The non-transitory computer-readable storage medium of claim 15, having additional instruction stored which, when executed by the computing device, cause the computing device to perform operations comprising:
receiving cart movement data, the cart movement data comprising force applied to the shopping cart handle and shopping cart speed;
determining weight of items placed in the cart;
determining work performed by movement of the cart.

17. The non-transitory computer-readable storage medium of claim 15, wherein in the baseline biometric data and the additional biometric data each respectively comprise a pulse of the user and a temperature of the user.

18. The non-transitory computer-readable storage medium of claim 15, wherein the biometric analysis comprises:
determining, based on the baseline biometric data, a baseline range;
recording multiple instances of the additional biometric data over time, to yield a table of biometric data;
identifying, within the table of biometric data, values which fall outside the baseline range; and
based on the values being outside the baseline range, determining that the check on the user should occur.

19. The non-transitory computer-readable storage medium of claim 18, further comprising determining a different user is using the shopping cart based on the values being outside the baseline range.

20. The non-transitory computer-readable storage medium of claim 18, wherein the multiple instances of the additional biometric data are received based upon a trigger condition being met at the shopping cart.
